(19) United States
(12) Patent Application Publication
(10) Pub. No.: US 2023/0310177 A1
(43) Pub. Date: Oct. 5, 2023

(54) POROUS INTERBODY IMPLANT

(71) Applicants: Brent FELIX, Salt Lake City, UT (US); INNOVASIS, INC., Salt Lake City, UT (US)

(72) Inventors: Brent FELIX, Salt Lake City, UT (US); Jeremy HEISER, Salt Lake City, UT (US); Mark WHITTINGTON, Highland, UT (US); Ron GROBERG, Syracuse, UT (US)

(21) Appl. No.: 18/129,130

(22) Filed: Mar. 31, 2023

Related U.S. Application Data
(60) Provisional application No. 63/326,246, filed on Mar. 31, 2022.

(51) Int. Cl. A61F 2/46; A61F 2/44

(52) U.S. Cl. CPC A61F 2/4611; A61F 2/4455

(57) ABSTRACT

An interbody implant may include a solid, unitary body and one or more porous layers. The interbody implant may include a homogenous interface between the one or more porous layers and a the solid, unitary body. The homogenous interface may fuse the material of the solid, unitary body to the one or more porous layers via a thermal process. The interbody implant may include a superior surface designed to abut an inferior surface of a vertebra and an inferior surface designed to abut a superior surface of a vertebra. The interbody implant may include a bone cavity extending between a first aperture and a second aperture of the superior and inferior surfaces.

### CROSS-REFERENCE TO RELATED APPLICATIONS

This application claims the benefit of U.S. Provisional Patent Application Ser. No. 63/326,246 entitled “POROUS INTERBODY IMPLANT,” filed on Mar. 31, 2022, the disclosure of which is incorporated herein by reference in its entirety.

### TECHNICAL FIELD

The present disclosure relates to spinal implants, methods, and devices. More specifically, the present disclosure relates to improved spinal implants, methods, and devices for spinal fusion in a patient.

### BACKGROUND

Spinal fusion procedures utilizing spinal implants can be used to correct spinal conditions such as degenerative disc disease, disc herniations, spondylolisthesis, stenosis, scoliosis, spinal deformities, or other spinal conditions through minimally invasive or invasive spinal surgery. For example, two or more vertebrae may be experiencing instability, deformity, or another abnormality which causes pain and discomfort to the patient. After preparatory surgery, a fusion cage containing bone graft material may be inserted into a disc space, thereby allowing bone to grow and connect the vertebrae, eventually resulting in bone fusion. The preparatory surgeries include multiple different types, each with advantages or disadvantages based on the patient's condition, surgeons' capabilities, or improvements to the field throughout the years. Among the various surgeries are Posterior Lumber Interbody Fusion (PLIF), Transforaminal Lumber Interbody Fusion (TLIF), Anterior Lumbar Interbody Fusion (ALIF), and Oblique Lateral Interbody Fusion (OLIF).

Unfortunately, despite significant advances and improvements in the field, interbody procedures with cage insertion may still result nerve damage, instrument malfunction, or non-fusion. Accordingly, improved surgical systems, methods, and devices that reduce or eliminate these postoperative outcomes would be desirable.

### SUMMARY

The various systems and methods of the present disclosure have been developed in response to the present state of the art, and in particular, in response to the problems and needs in the art that have not yet been fully solved by currently available surgical instruments, devices, systems, and methods for implanting bone anchor assemblies in a patient.

According to some embodiments, an interbody implant may be used to fuse a superior vertebra and an inferior vertebra of a patient. The interbody implant may have a body with a solid, unitary structure. The body may define a superior side shaped to abut an inferior end plate of the superior vertebra, the superior side defining a first aperture, an inferior side shaped to abut a superior end plate of the inferior vertebra, the inferior side defining a second aperture, a leading end, a trailing end, a first lateral side, a second lateral side, and a bone growth cavity extending between the first aperture and the second aperture. The interbody implant may further include one or more porous layers that are secured to the superior side, the inferior side, the first lateral side, and the second lateral side by a homogenous interface between the body and the porous layer.

Each of the one or more porous layers may have a web-like scaffold having curved surfaces that define substantially spherical voids, the substantially spherical voids being interconnected at tangent points.

The web-like scaffold may have a material formed from a substantially uniform mixture of bone growth material and thermoplastic polymer.

The one or more porous layers may be a single porous layer that extends across the inferior side, the first lateral side, the superior side, and the second lateral side.

The one or more porous layers may terminate short of the leading end.

The leading end may include a solid bumper positioned to protect the one or more porous layers from abrasion against bone during insertion of the interbody implant between the superior vertebra and the inferior vertebra.

The leading end may include an exterior surface that is flush with adjoining exterior surfaces of the one or more porous layers.

At least one of the one or more porous layers may have a variable thickness.

At least one of the one or more porous layers may have a flat interior surface facing and secured to the superior side, the inferior side, the first lateral side, or the second lateral side, and a convex exterior surface.

The one or more porous layers may include a superior layer secured to the superior side, an inferior layer secured to the inferior side, a first lateral layer secured to the first lateral side, and a second lateral layer secured to the second lateral side. The body may include solid structures extending longitudinally between each of the superior layer, the inferior layer, the first lateral layer, and the second lateral layer.

The body may have a solid structure on the trailing end, the solid structure defining a non-porous interface that facilitates attachment of the interbody implant to an implant inserter. The one or more porous layers may extend to cover superior, inferior, and/or lateral aspects of the trailing end.

The one or more porous layers may further be secured to inwardly-facing surface of the bone growth cavity.

At least one of the superior side, the inferior side, the first lateral side, and the second lateral side may have a boss extending through the one or more porous layers.

According to some embodiments, an interbody implant may be used to fuse a superior vertebra and an inferior vertebra of a patient. The interbody implant may have a body with a solid, unitary structure. The body may define a superior side shaped to abut an inferior end plate of the superior vertebra, the superior side defining a first aperture, an inferior side shaped to abut a superior end plate of the inferior vertebra, the inferior side defining a second aperture, a leading end, a trailing end, a first lateral side, a second lateral side, and a bone growth cavity extending between the first aperture and the second aperture. The interbody implant may further include one or more porous layers that are secured to the superior side and the inferior side. At least one of the one or more porous layers may have a variable thickness.

At least one of the one or more porous layers may have a flat interior surface facing and secured to the superior side or the inferior side, and a convex exterior surface.

At least one of the one or more porous layers may have a leading edge at which the variable thickness approaches zero.

Each of the one or more porous layers may be formed of a substantially uniform mixture of bone growth material and thermoplastic polymer.

According to some embodiments, an interbody implant may be used to fuse a superior vertebra and an inferior vertebra of a patient. The interbody implant may have a body with a solid, unitary structure. The body may define a superior side shaped to abut an inferior end plate of the superior vertebra, the superior side defining a first aperture, an inferior side shaped to abut a superior end plate of the inferior vertebra, the inferior side defining a second aperture, a leading end, a trailing end, a first lateral side, a second lateral side, and a bone growth cavity extending between the first aperture and the second aperture. The interbody implant may further include one or more porous layers that are secured to the superior side and the inferior side. The leading end may include a solid bumper positioned to protect the one or more porous layers from abrasion against bone during insertion of the interbody implant between the superior vertebra and the inferior vertebra. The body may have a solid structure on the trailing end, the solid structure defining a non-porous interface that facilitates attachment of the interbody implant to an implant inserter. The one or more porous layers may extend to cover superior, inferior, and/or lateral aspects of the trailing end.

At least one of the one or more porous layers may have a variable thickness.

Each of the one or more porous layers may be formed of a substantially uniform mixture of bone growth material and thermoplastic polymer.

These and other features and advantages of the present disclosure will become more fully apparent from the following description and appended claims, or may be learned by the practice of the systems and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1A is a perspective view of an interbody implant, according to one embodiment;

FIG. 1B is an alternative perspective view of the interbody implant of FIG. 1A;

FIG. 2A is another alternative perspective view of the interbody implant of FIG. 1A;

FIG. 2B is another alternative perspective view of the interbody implant of FIG. 1A;

FIG. 3A is a top view of the interbody implant of FIG. 1A;

FIG. 3B is a bottom view of the interbody implant of FIG. 1A;

FIG. 4A is front elevation view of the interbody implant of FIG. 1A;

FIG. 4B is a rear elevation view of the interbody implant of FIG. 1A;

FIG. 5A is a left elevation view of the interbody implant of FIG. 1A;

FIG. 5B is a right elevation view of the interbody implant of FIG. 1A;

FIG. 5C is a left elevation, section view of the interbody implant of FIG. 1A;

FIG. 5D is an exploded view of a homogeneous interface between a porous layer and a solid portion of the implant of FIG. 1A;

FIG. 5E is an exploded view of a scaffold of the homogeneous interface of FIG. 5D;

FIG. 6A is a perspective view of an interbody implant, according to another embodiment;

FIG. 6B is an alternative perspective view of the interbody implant of FIG. 6A;

FIG. 7A is a perspective view of an interbody implant, according to another embodiment;

FIG. 7B is a right elevation view of the interbody implant of FIG. 7A;

FIG. 7C is a perspective view of a body of the interbody implant of FIG. 7A without the porous layer;

FIG. 8A is a perspective view of an interbody implant, according to another embodiment;

FIG. 8B is an alternative perspective view of the interbody implant of FIG. 7A;

FIG. 9A is a perspective view of an interbody implant, according to another embodiment;

FIG. 9B is an alternative perspective view of the interbody implant of FIG. 8A;

FIG. 10A is a perspective view of an interbody implant, according to another embodiment;

FIG. 10B is an alternative perspective view of the interbody implant of FIG. 9A;

FIG. 10C is another alternative perspective view of the interbody implant of FIG. 9A, in comparison with another interbody implant such as the interbody implant of FIG. 1A;

FIG. 11A is a perspective view of an interbody implant, according to another embodiment;

FIG. 11B is a side elevation view of the interbody implant of FIG. 11A;

FIG. 11C is a rear elevation view of the interbody implant of FIG. 11A;

FIG. 11D is a front elevation view of the interbody implant of FIG. 11A;

FIG. 12A is a perspective view of an interbody implant, according to another embodiment;

FIG. 12B is an alternative perspective view of the interbody implant of FIG. 12A;

FIG. 12C is a side elevation view of the interbody implant of FIG. 12A;

FIG. 12D is another alternative perspective view of the interbody implant of FIG. 12A;

FIG. 13A is a perspective view of an interbody implant, according to another embodiment;

FIG. 13B is a side elevation view of the interbody implant of FIG. 13A;

FIG. 14A is a perspective view of an interbody implant, according to another embodiment;

FIG. 14B is a left elevation view of the interbody implant of FIG. 14A;

FIG. 14C is an exploded left elevation view of the assembly of the interbody implant of FIG. 14A;

FIG. 15A is a perspective view of an interbody implant, according to another embodiment;

FIG. 15B is a perspective view of the body of the interbody implant of FIG. 15A without the porous layer;

FIG. 16A is a perspective view of an interbody implant, according to another embodiment;

FIG. 16B is a perspective view of the body of the interbody implant of FIG. 16A without the porous layer;

FIG. 17A is a perspective view of an interbody implant, according to another embodiment;

FIG. 17B is a right elevation view of the interbody implant of FIG. 17A;

FIG. 17C is a perspective view of the body of the interbody implant of FIG. 17A without the porous layer;

FIG. 18A is a perspective view of an interbody implant, according to another embodiment;

FIG. 18B is a right elevation section view of the interbody implant of FIG. 18A;

FIG. 18C is a perspective view of the body of the interbody implant of FIG. 18A without the porous layer;

FIG. 19A is a perspective view of an interbody implant, according to another embodiment;

FIG. 19B is a top view of the interbody implant of FIG. 19A;

FIG. 19C is a front elevation view of the interbody implant of FIG. 19A;

FIG. 19D is a left elevation view of the interbody implant of FIG. 19A;

FIG. 19E is a rear elevation view of the interbody implant of FIG. 19A;

FIG. 19F is a left section view of the interbody implant of FIG. 19A;

FIG. 20A is a perspective view of an interbody implant, according to another embodiment;

FIG. 20B is a top view of the interbody implant of FIG. 20A;

FIG. 20C is a front elevation view of the interbody implant of FIG. 20A;

FIG. 20D is a left elevation view of the interbody implant of FIG. 20A; and

FIG. 20E is a rear elevation view of the interbody implant of FIG. 20A.

It is to be understood that the drawings are for purposes of illustrating the concepts of the disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the Figures herein, could be arranged and designed in a wide variety of different configurations. Thus, the following more detailed description of the embodiments of the apparatus and method, as represented in the Figures, is not intended to limit the scope of the present disclosure, as claimed in this or any other application claiming priority to this application, but is merely representative of exemplary embodiments of the present disclosure.

Standard medical directions, planes of reference, and descriptive terminology are employed in this specification. For example, anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. Ipsilateral means on the same side of the body. Contralateral means on the opposite side of the body. A sagittal plane divides a body into right and left portions. A midsagittal plane divides the body into bilaterally symmetric right and left halves. A coronal plane divides a body into anterior and posterior portions. A transverse plane divides a body into superior and inferior portions. These descriptive terms may be applied to an animate or inanimate body.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Described herein are implants for spinal surgery and/or correction, including but not limited to Posterior Lumber Interbody Fusion (PLIF), Transforaminal Lumber Interbody Fusion (TLIF), Anterior Lumbar Interbody Fusion (ALIF), and Oblique Lateral Interbody Fusion (OLIF). The geometry of the implant, surface area of porous surfaces, thickness of porous surfaces, lordotic angle of the implant, structure, shape, size, and other characteristics of the implant may be tailored to the intended use.

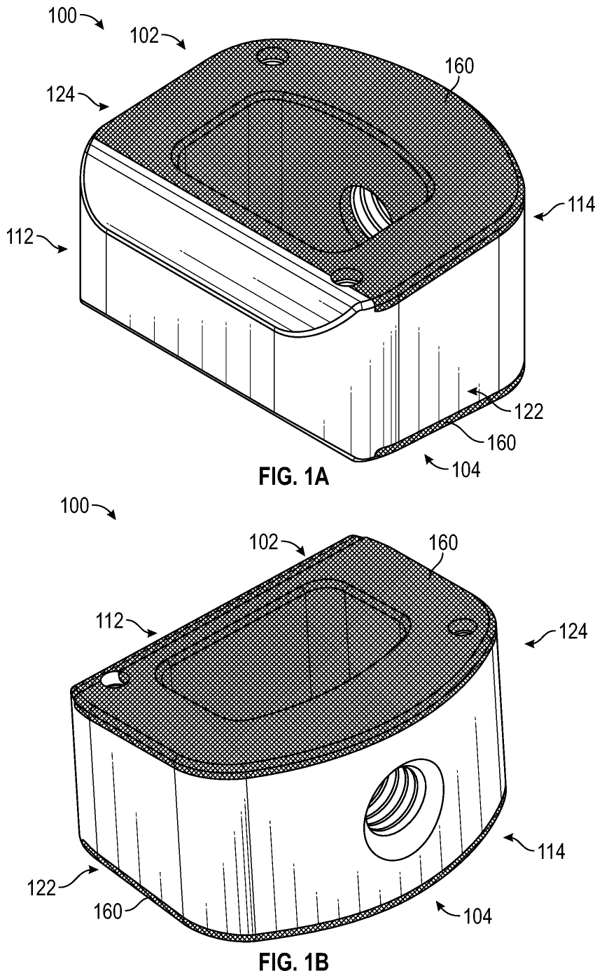

FIGS. 1A and 1B illustrate perspective views of an interbody implant, or implant 100, according to an embodiment of the present disclosure. The implant 100 may generally include a core or a body portion and a structurally modified portion, which together have a superior side 102, an inferior side 104, a leading end 112, a trailing end 114, a first lateral side 122, and a second lateral side 124. The implant 100 may be shaped for insertion into the space between a superior vertebra and an inferior vertebra (not shown). Upon insertion, the superior side 102 may abut the inferior end plate of the superior vertebra, while the inferior side 104 may abut the superior end plate of the inferior vertebra. The superior side 102 may be angled nonparallel to the inferior side 104 in order to provide the desired lordotic angle to the space between the superior and inferior vertebrae. In alternative embodiments, the superior side 102 may be parallel to the inferior side 104, or may even be angled to provide kyphosis.

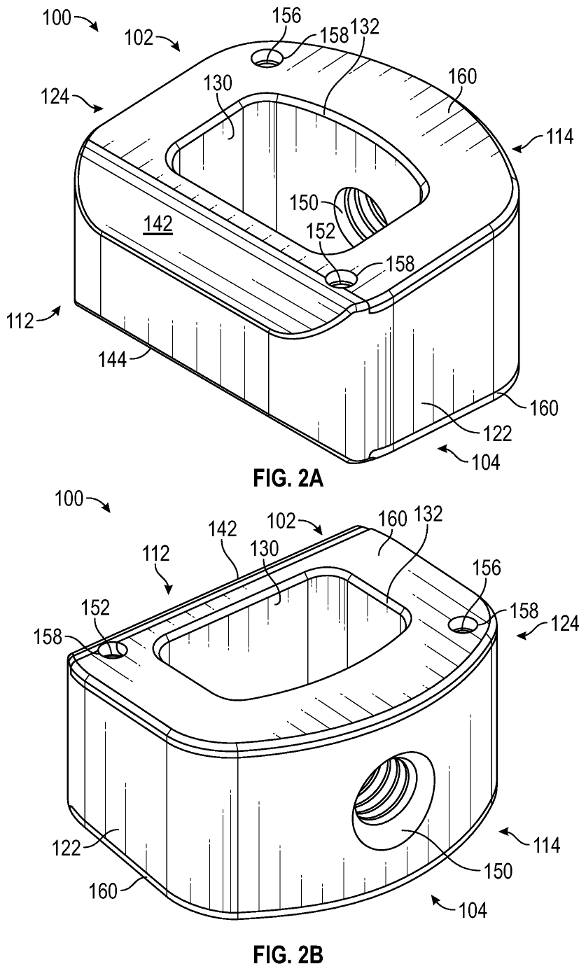

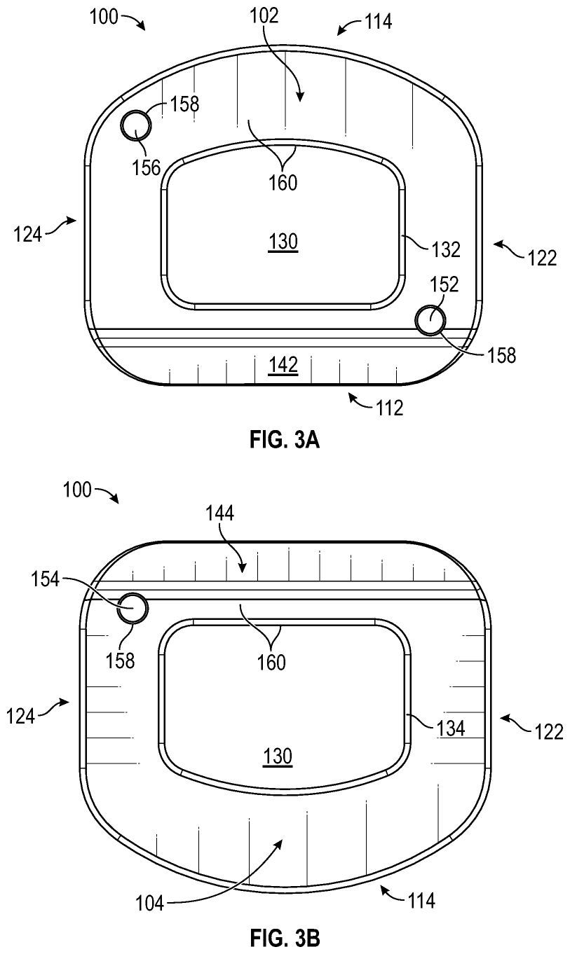

FIGS. 2A and 2B illustrate alternate perspective views of the implant 100. The implant 100 may further include a cavity 130 extending between the superior side 102 and the inferior side 104. The cavity 130 may open on the superior side 102 at a first aperture 132 (e.g., superior aperture), and on the inferior side 104 at an second aperture 134 (e.g., inferior aperture—shown in FIG. 3B). in some embodiments, the cavity 130 may be a bone growth cavity, being sized and shaped to permit growth of a bone column between the superior vertebra and the inferior vertebra. The bone column may permit the superior vertebra and the inferior vertebra to essentially fuse together. In alternative embodiments, the implant 100 may be solid, or may have multiple cavities extending between the superior side 102 and the inferior side 104 to permit growth of multiple bone columns between the superior and inferior vertebrae.

Optionally, the implant 100 may be combined with other implants (not shown), such as bone plates, pedicle screw and rod systems, another fusion cage (e.g., identical or dissimilar), and/or the like in order to further immobilize the joint defined between the superior and inferior vertebrae while fusion occurs. In the alternative, the implant 100 may be a “standalone” implant designed to function independently of other implants.

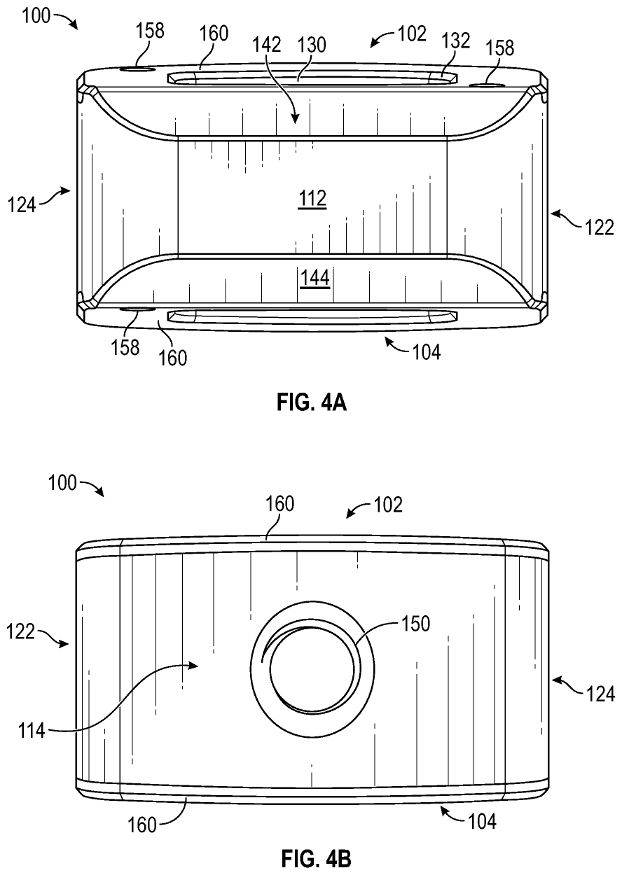

FIGS. 3A through 5B illustrate additional views of the implant 100. The leading end 112 of the implant 100 may have one or more features that facilitate insertion of the implant 100 between the superior and inferior vertebrae. For example, the leading end 112 may have a superior taper 142 adjacent to the superior side 102, and an inferior taper 144 adjacent to the inferior side 104. The superior taper 142 and the inferior taper 144 may cooperate to reduce the height of the leading end 112, relative to the trailing end 114 and/or the central portion of the implant 100, making it easier to insert the implant 100 between the superior and inferior vertebrae. The angles of the superior taper 142 and the inferior taper 144 may be selected such that the leading end 112 can distract the superior and inferior vertebrae during insertion so that the intervertebral space can receive the implant 100 in its entirety.

The trailing end 114 of the implant 100 may have one or more inserter connection features. In the exemplary embodiment of FIG. 1A, the trailing end 114 may have a threaded hole 150 (see, FIGS. 1B, 2B, and 4B) that can receive a corresponding threaded post (not shown) of an inserter. After insertion between the superior and inferior vertebrae, the threaded post may be rotated out of engagement with the threaded hole 150 so that the inserter can be detached from the implant 100. The threaded hole 150 is only one of many inserter connection features that may be used. In alternative embodiments, the implant 100 may have other inserter connection features (not shown), such as pockets, grooves, or protrusions, that mate with corresponding features on the inserter. Such an inserter may have moving parts such as a threaded post that rotates into engagement with the threaded hole 150, or jaws that pinch together to grip the implant 100.

The implant 100 may further have one or more features that facilitate visualization of the implant 100 under X-ray, fluoroscopy, and/or other imaging techniques. For example, the implant 100 may have a first radiographic marker 152, a second radiographic marker 154, and a third radiographic marker 156. The first radiographic marker 152, the second radiographic marker 154, and the third radiographic marker 156 may be made of a biocompatible, radio-opaque material that will be visible under fluoroscopy. For example, the first radiographic marker 152, the second radiographic marker 154, and the third radiographic marker 156 may be made of Titanium or the like. The first radiographic marker 152, the second radiographic marker 154, and the third radiographic marker 156 may be formed in any shape, including but not limited to spheres, rods, and pins, and may be vertically-oriented pins in the embodiment of FIGS. 1A through 5C. In some embodiments more or fewer than three radiographic markers may be used, and may be arranged and/or oriented differently from the arrangement shown in these drawings.

As shown, the first radiographic marker 152, the second radiographic marker 154, and the third radiographic marker 156 may all extend superior-inferiorly. The first radiographic marker 152 and the second radiographic marker 154 may be proximate the leading end 112, and the third radiographic marker 156 may be proximate the trailing end 114. As shown, the first radiographic marker 152 and the third radiographic marker 156 may be inserted through holes 158 in the superior side 102, and the second radiographic marker 154 may be inserted through a hole 158 in the inferior side 104. A diameter of the holes (e.g., hole 158) in a porous layer 160 (discussed below) of the implant 100 may be larger than a diameter of the holes in the remaining portion of the body of the implant 100 to facilitate ease of insertion of the radiographic markers.

At least some surfaces of the implant 100 may be designed to facilitate bone in-growth, through-growth, and/or bone on-growth with the implant 100. For example, one or more surfaces of the implant 100 may be porous to promote growth of bone into the surfaces, thereby enhancing adherence of the implant 100 to the superior and/or inferior vertebrae and/or bone growth between the superior and inferior vertebrae. The pores may be large and/or small (for example, nanoscale).

According to some examples, at least part of the implant 100 may be formed of a structurally modified (e.g., porous), biocompatible material such as a porous PEEK, Titanium, Cobalt Chromium, or the like. The pores may have a wide range of sizes, for example, from 1 μm to 10,000 μm, or more precisely from 10 μm to 5,000 μm, or even more precisely from 100 μm to 1,000 μm.

In alternative embodiments, the pores may have a narrow range of sizes. For example, the pores may range in size from 200 μm to 600 μm, or more precisely, from 300 μm to 500 μm, or even from 350 μm to 400 μm. If desired, the pores may be formed in a pattern such that the pores are connected at tangent points within the material. Thus, not only pores adjacent to the surface of the material, but also pores underneath the surface may be available for bone in-growth. In some embodiments, methods such as those set forth in U.S. Pat. No. 10,485,897, which is incorporated herein by reference, may be used to generate such pores. The pores may exist in a relatively even pattern (i.e., a “matrix”), rather than a random arrangement.

The pores may have any shape. In some embodiments, they may be spherical. In alternative embodiments, their shapes may be oblong, trapezoidal, rectangular prisms, pyramidal, conical, or the like. Any combination of rectilinear and/or organic shapes may be used.

In embodiments having spherical pores, the structural portion of the porous material comprises a scaffold of curved surfaces, having spherical voids that are substantially interconnected at tangent points of the spherical voids and/or curved surfaces. Generally, (meaning not limited to only spherical pore embodiments), the pores may have a wide range of interconnectivity, for example, from 40% to 90% interconnectivity, or more precisely from 50% to 80% interconnectivity, or even more precisely from 60% to 70% interconnectivity.

In some embodiments, the entirety of the implant 100 may have the same porous structure (for example, porous PEEK). In alternative embodiments, the implant 100 may exist as a combination of solid and porous portions (for example, solid PEEK and porous PEEK), with the porous portion defining a superficial layer on one or more surfaces of the implant 100. Such a superficial layer may have a thickness within the range of 0.01 mm to 5 mm, or more precisely, from 0.1 mm to 2 mm, or yet more precisely, from 0.2 mm to 1 mm, or still more precisely, from 0.3 mm to 0.6 mm.

Such a porous layer may exist only on bone-facing surfaces of the implant 100 (for example, on the superior side 102 and the inferior side 104, or more specifically on the portions of the superior side 102 and the inferior side 104 that will be in direct contact with the bone of the superior and inferior vertebrae. In the alternative, such a porous layer may wrap around to additional surfaces of the implant 100. This is the configuration shown in FIGS. 1A through 5C, in which a porous layer 160 is present on the superior side 102 and the inferior side 104, but also wraps around through the superior aperture 132 and the inferior aperture 134 to cover the potentially vertical inwardly-facing surfaces of the cavity 130. Thus, the porous layer 160 may cover not just the surfaces that will be in direct contact with the superior and inferior vertebrae at the time of implantation, but also the surfaces that will be in contact with the bone column to be formed to fuse the superior and inferior vertebrae together.

In the exemplary embodiment of FIGS. 1A through 5C, the porous layer 160 may cover the superior side 102 and the inferior side 104, except for the portion that approaches the leading end 112 and defines the superior taper 142 and the inferior taper 144. The superior taper 142 and the inferior taper 144 may remain solid so that they can slide against the superior and inferior vertebrae and/or remain uncompressed in response to pressure urging them against the superior and inferior vertebrae. The porous layer 160 may optionally extend across the superior taper 142 and the inferior taper 144, but care may need to be taken not to crush the pores of the superior taper 142 and the inferior taper 144 during insertion into the space between the vertebrae.

Further, in the exemplary embodiment of FIGS. 1A through 5C, the portions of the superior taper 142 and the inferior taper 144 adjacent to the porous layer 160 may define a superior solid bumper 162 and an inferior solid bumper 164, respectively, that lead the porous layer 160 (on the superior side 102 and the inferior side 104, respectively), into the space between the vertebrae, and thus serve to protect the porous layer 160 against damage during insertion. The superior solid bumper 162 and the inferior solid bumper 164 may serve to distract and/or abrade away bone, during insertion, that could otherwise compress and disturb the porosity of the porous layer 160.

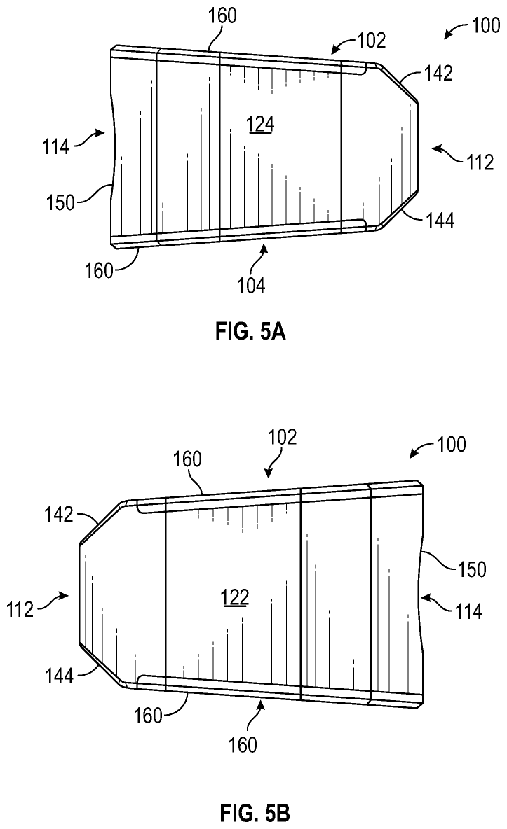

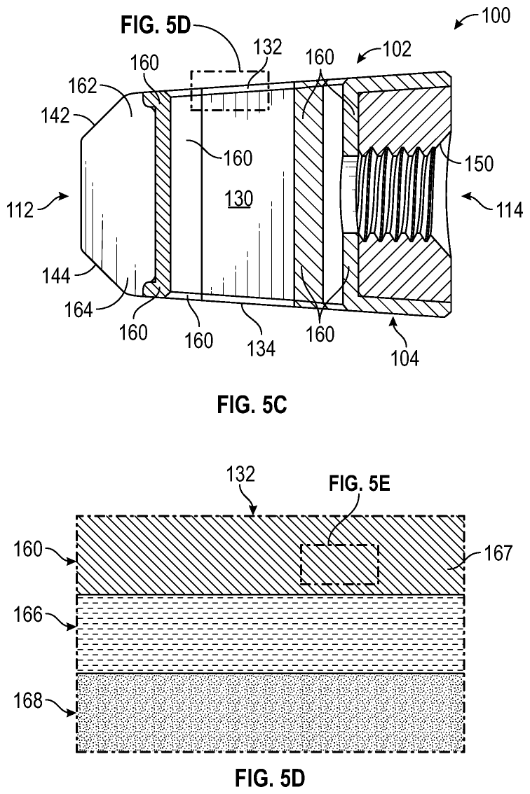

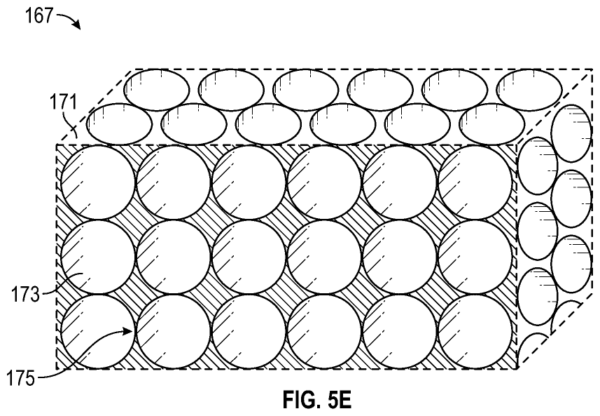

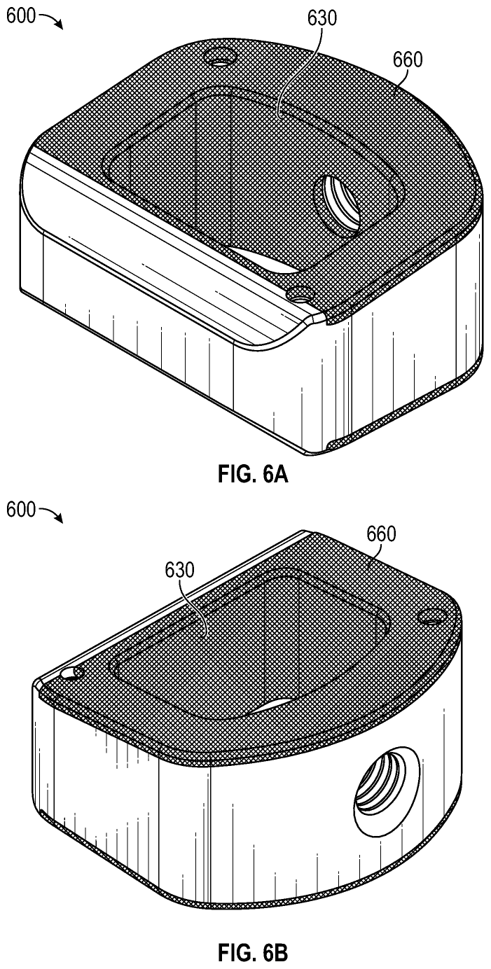

FIG. 5C illustrates a section view of the implant 100. In addition to covering the stated portions of the superior side 102 and the inferior side 104, the porous layer 160 may extend through the superior aperture 132 and the inferior aperture 134 to cover the interior surfaces of the cavity 130. This feature of some embodiments of the porous layer is also depicted in FIGS. 6A and 6B, where the porous layer 660 extends through apertures to cover surfaces of the cavity 630. Thus, the porous layer 160 and/or 660 may also face, and facilitate formation of, the bone column that is to be formed through the cavity 130 and/or 630 of the implant 100 and/or 600.

In addition to or in the alternative to the porous layer 160, one or more surfaces of the implant 100 may be coated and/or infused with an osteogenic substance designed to promote bone growth. For example, various calcium phosphates may be used, including hydroxyapatite (“HA”). Such materials may be provided as a surface layer or coating, or may be seated deeper in a porous structure. The osteogenic coating or infusion may facilitate and/or enhance the osseointegration process during the early stages of healing.

In some embodiments, where the osteogenic material is applied as a coating, the coating may be applied to the entire exterior of the implant 100. In the alternative, such a coating may be applied only to the porous layer 160 of the implant 100. The thickness of the coating may be within the range of 0.001 μm to 1 μm in thickness, or more precisely, 0.01 μm to 0.1 μm, or yet more precisely, from 0.015 μm to 0.05 μm. In some embodiments, the thickness of the coating may be about 0.02 μm (20 nm). Use of such a thin coating may help to preserve the porosity of the porous layer 160 (or in alternative embodiments, the porosity of the entire implant), while still providing the osteogenic properties mentioned above. The thin coating may additionally or alternatively eliminate at least some of the risks associated with thicker osteogenic coatings, such as poor coating integration and poor mechanical stability.

In addition to or in the alternative to a coating, the osteogenic material may be incorporated into the material of the cage. For example, the implant 100 may be made of HA PEEK, or a combination of HA PEEK and porous PEEK and/or porous HA PEEK.

Additionally or alternatively, the osteogenic material may be infused into the material of which the implant 100 is formed, or the porous portion thereof. Osteogenic material may consist of, but is not limited to: Hydroxyapatite (HA), Sintered and/or Unsintered calcium phosphate compound, Amorphous CaP (ACP), Biphasic CaP (BCP), Tetracalcium Phosphate (TTCP), Dicalcium Phosphate Anhydrous (DCPA), Dicalcium Phosphate Dihydrate (DCPD), Tricalcium Phosphate (TCP), Alpha-Tricalcium Phosphate (alpha-TCP), Beta-Tricalcium phosphate (beta-TCP), and/or combinations of the foregoing. The osteogenic material could also consist of a substituted HA, TCP, & BCP; wherein each substituted CaP can be synthesized with some of the atoms of Ca or some of the PO₄ molecules replaced by other elements or molecules to bring about certain desirable biologic responses. Calcium phosphate compounds can have substitution of F, Ag, Sr, Mg, Zn and CO₃. For example, the implant 100 may be formed of porous PEEK infused with HA. The HA may be distributed uniformly or substantially uniformly throughout the porous PEEK material, making it a porous PEEK HA structure. As another example, the implant 100 may be formed of a combination of solid and porous PEEK materials, with the PEEK existing as a layer such as porous layer 160, in which HA is infused only throughout the porous layer 160. In yet another example, the implant 100 may be formed of two separate portions. The first portion is a combination of HA and PEEK (e.g., HA distributed uniformly throughout the PEEK), neither of which is porous. The second portion is also a combination of HA and PEEK, formed together with porogen (e.g., spherical salt particles), which are subsequently leached out, making the second portion porous. The two portions may then be fused together by applying heat sufficient to melt at least one of the two portions to the other of the two portions. In some embodiments, both portions include an organic polymer or an organic thermoplastic polymer, such as s polyaryletherketone (PAEK), PEEK, polyethylene glycol (PEG), polvinylpyrrolidone (PVP), polyvinyl alcohol (PVA), or combinations thereof.

In some embodiments, the osteogenic material is inserted into the cavity 130 before the implant 100 is inserted into the patient. Additionally or alternatively, osteogenic material may be placed around the implant 100 after implantation. The osteogenic material may be inserted as a fluid, a viscous fluid, a non-Newtonian fluid, a putty, or combinations thereof. The osteogenic material may include HA, tricalcium phosphate (TCP), biphasic calcium phosphate, polymethylmethacrylate (PMMA), monocalcium phosphate, calcium carbonate, calcium sulphate, or combinations thereof.

FIG. 5D is an exploded view of a portion of the surface forming the superior aperture 132 of the implant 100. In some embodiments, the exploded portion of the surface forming the superior aperture 132 may include the porous layer 160, a homogenous interface 166, and a non-porous interface 168 of the solid body core of the implant 100.

In some embodiments, the homogeneous interface 166 includes a material of the porous layer 160 fused to the non-porous interface 168 of the body core as a result of a thermal process. For example, referring now to FIG. 5E, the porous material 167 may include a web-like scaffold 171 comprising a thermoplastic material, such as a PEEK/HA composite having pores formed therein as a result of leaching spherical salt particles from the PEEK/HA composite. The web-like scaffold 171 may have curved surfaces that define substantially spherical voids 173. The substantially spherical voids 173 may be interconnected at tangent points 175. The tangent points 175 may be found at apexes of the curved surfaces that define the substantially spherical voids 173. The non-porous interface 168 of the solid body core may comprise a PEEK/HA composite. The porous material 167 resulting from the leaching process may be heated as it is in contact with the PEEK/HA composite comprising the non-porous interface 168. The heating may include melting the portion of the porous material 167 that is in contact with the non-porous interface 168, thereby fusing the porous material 167 to the non-porous interface 168. In other embodiments, the non-porous interface 168 is the portion that is heated and melted to fuse it to the porous material 167. In yet other embodiments, both the non-porous interface 168 and the porous material 167 are heated and portions of both are melted to fuse the materials together and form the homogeneous interface 166.

Many different combinations of porosity and osteogenic material may be used within the scope of the present disclosure. Four exemplary combinations will be presented below:

### Example 1—Porous PEEK Spinal Cage that is Coated with a Nano-Thick Layer of HA

Porous PEEK Spinal Cage
Pore size ranging from 200-600 um
Pores are interconnected at tangent points within the PEEK material
Cage configuration options:
   Cage may be constructed of a combination of Solid PEEK and Porous PEEK, wherein Porous PEEK is incorporated as a superficial layer (up to 3 mm thick)
   Cage may be constructed of a combination of Solid PEEK and Porous PEEK, wherein the Porous PEEK extends from one face of the cage to another
   Cage may be constructed entirely of Porous PEEK The entire cage is then coated with a nano-thick layer of Hydroxyapatite (HA).
HA coating facilitates and enhances the osseointegration process during the early phases of healing
HA coating to be less than 0.1 μm (100 nm), preferably 0.02 μm (20 nm)
Nano-coating to preserve the Porous PEEK features
Nano-coating thickness eliminates the risks associated with thicker HA coatings; including, poor coating integration and poor mechanical stability.

### Example 2—Porous PEEK/HA PEEK Spinal Cage Coated with a Nano-Thick Layer of HA

Porous PEEK/HA PEEK Spinal Cage
Pore size ranging from 200-600 um
Pores are interconnected at tangent points within the PEEK material
Configuration options:
   Cage may be constructed of a combination of Solid HA PEEK and Porous PEEK, wherein Porous PEEK is incorporated as a superficial layer (up to 3 mm thick)
   Cage may be constructed of a combination of Solid HA PEEK and Porous PEEK, wherein the Porous PEEK extends from one face of the cage to another
The entire cage is then coated with a nano-thick layer of Hydroxyapatite (HA).
HA coating facilitates and enhances the osseointegration process during the early phases of healing
HA coating to be less than 0.1 μm (100 nm), preferably 0.02 μm (20 nm)
Nano-coating to preserve the Porous PEEK features
Nano-coating thickness eliminates the risks associated with thicker HA coatings; including, poor coating integration and poor mechanical stability.
The HA Coated Porous PEEK allows for early bone growth into the porous layer and the HA infused Solid elements provide the long-term benefits of the infused HA PEEK
The Porous PEEK/HA PEEK with HA nano coating would allow for improved manufacturing capabilities and more intricate designs

### Example 3—Porous PEEK Spinal Cage that is Infused with HA

Porous HA PEEK Spinal Cage
Pore size ranging from 200-600 um
Pores are interconnected at tangent points within the PEEK material
HA is distributed throughout the porous PEEK material, making it a Porous HA PEEK structure
Configuration options:
   Cage may be constructed of a combination of Solid HA PEEK and Porous HA PEEK, wherein Porous HA PEEK is incorporated as a superficial layer (up to 3 mm thick)
   Cage may be constructed of a combination of Solid HA PEEK and Porous HA PEEK, wherein the Porous HA PEEK extends from one face of the cage to another
   Cage may be constructed entirely of Porous HA PEEK

### Example 4—Porous PEEK Spinal Cage that is Infused with HA

Cage is constructed of a combination of Solid HA PEEK and Porous HA PEEK, wherein Porous HA PEEK is incorporated as a superficial layer (up to 3 mm thick)
Porous Layer Thickness: 0.3-3.0 mm
Porous Layer may be on one or more surfaces of the cage.
Porous Layer on the superior and/or inferior endplate contact surfaces, the vertical wall of the graft window, and/or on the surfaces surrounding the superior and/or inferior apertures that provide access to the graft window
HA is distributed throughout the porous PEEK layer, making it a Porous HA PEEK structure
Pore size ranging from 200-600 um
Pore Size: 0.3-0.425 mm (300-425 um)
Pores are interconnected at tangent points within the HA PEEK material

The shape of the implant 100 is merely exemplary. Implants according to the present disclosure may be made in a wide variety of shapes and sizes. FIGS. 6A through 9C present examples of different shapes and sizes.

FIG. 6A is a perspective view of an interbody implant, or implant 600, according to another embodiment. FIG. 6B is an alternative perspective view of the implant 600 of FIG. 6A. The implant 600 may have a configuration that is generally similar to that of the implant 100, but may be in a larger size. The implant 600 may also have a cavity 630 that is thus proportionately larger than the cavity 130 of the implant 100. As mentioned above in connection with the implant 100, the implant 600 may alternatively have no cavity, or multiple cavities arranged to provide structural and/or load-bearing constructs in suitable locations of the implant 600. The implant 600 may have a porous layer 660 facing the superior and inferior vertebrae, and optionally covering the inwardly-facing surfaces of the cavity 630.

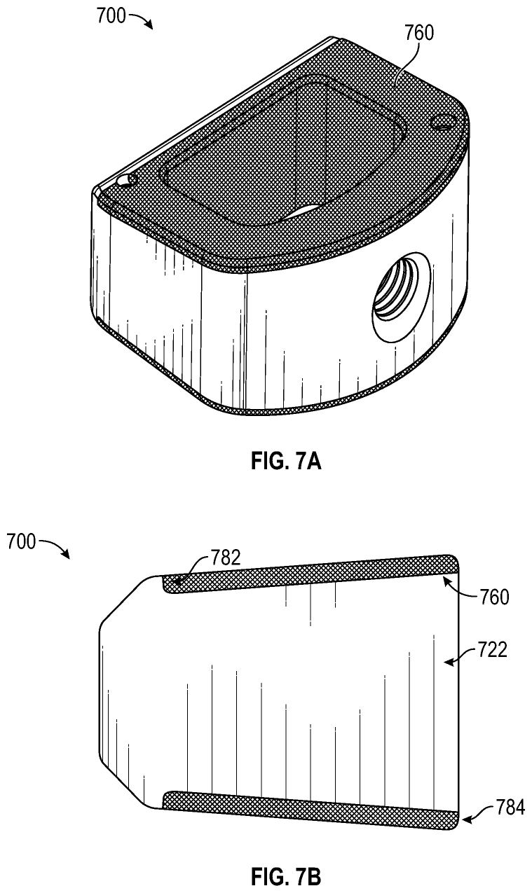

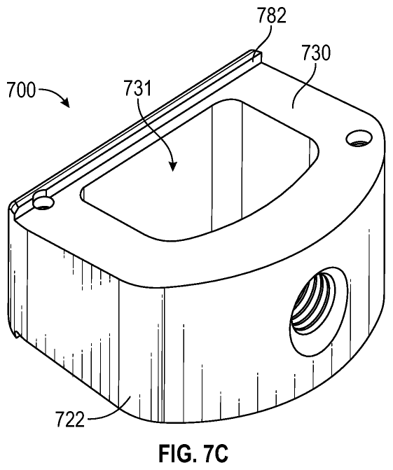

FIG. 7A is a perspective view of an interbody implant, or implant 700, according to another embodiment. FIG. 7B is a side elevation view, depicting the first lateral side 722 of the implant 700 of FIG. 7A. FIG. 7C is a perspective view of a body of the implant 700 without the porous layer 760. The implant 700 may be in a smaller in size than the implant 100 and the implant 600 (e.g., such as would be used in children), and may have a cavity 730 that similarly promotes fusion between the superior and inferior vertebrae. The implant 700 may have a porous layer 760 facing the superior and inferior vertebrae, and coating the inwardly-facing surfaces 731 of the cavity 730. In some embodiments, the inwardly-facing surfaces 731 may include non-linear surfaces. In other embodiments, the inwardly-facing surfaces 731 may include linear surfaces. In yet other embodiments, the inwardly-facing surfaces may include both linear and non-linear surfaces 731.

The implant 700 may have a superior exterior surface 782 and/or inferior exterior surface (not shown) that abuts an adjoining exterior surface of the porous layer 760. The superior exterior surface 782 is a portion of a bumper structure (e.g., similar or identical, except in size, to superior solid bumper 162). The implant 700 may further include rounded, beveled, or chamfered corners and/or edges 784. The angle or radius of curvature of the bevel/chamfer of the corners and/or edges 784 may be increased or decreased during manufacture of the implant 700, depending on the intended use, desired amount of friction, angle of insertion of the implant, manufacture technique and/or technology used to manufacture the implant 700, or combinations thereof.

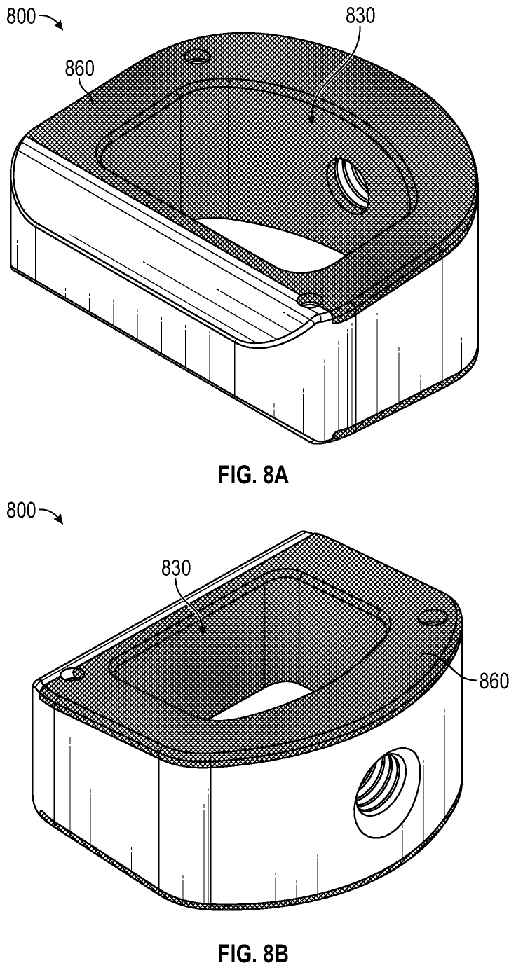

FIG. 8A is a perspective view of an interbody implant, or implant 800, according to another embodiment. FIG. 8B is an alternative perspective view of the implant 800 of FIG. 8A. The implant 800 may be in a larger size than the implant 100 and the implant 600, and may have a cavity 830 that similarly promotes fusion between the superior and inferior vertebrae. The implant 800 may have a porous layer 860 facing the superior and inferior vertebrae, and optionally, covering the inwardly-facing surfaces of the cavity 830.

FIG. 9A is a perspective view of an interbody implant, or implant 900, according to another embodiment. FIG. 9B is an alternative perspective view of the implant 900 of FIG. 9A. The implant 900 may be in a larger size than the implant 100, the implant 600, and the implant 700, and may have a cavity 930 that similarly promotes fusion between the superior and inferior vertebrae. The implant 900 may have a porous layer 960 facing the superior and inferior vertebrae, and optionally, covering the inwardly-facing surfaces of the cavity 930.

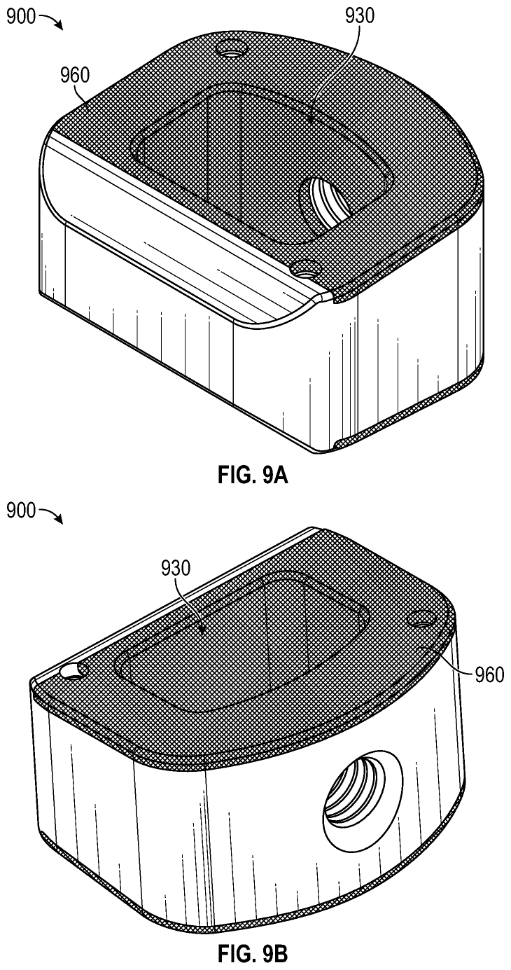

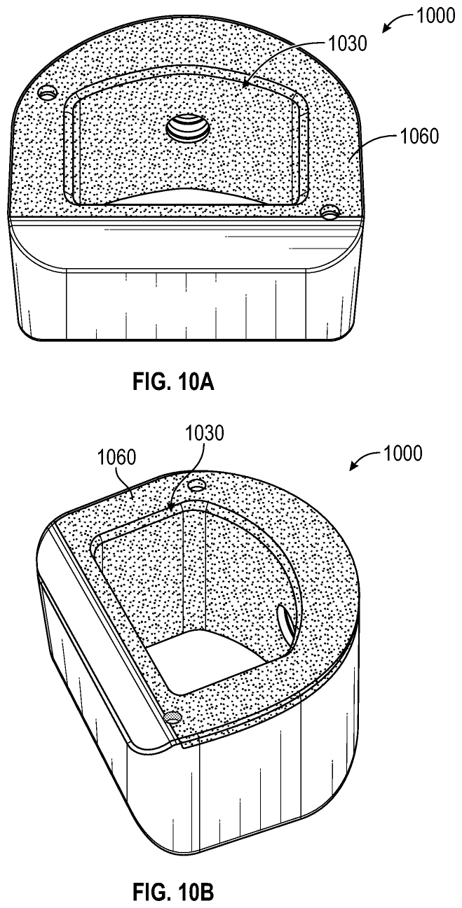

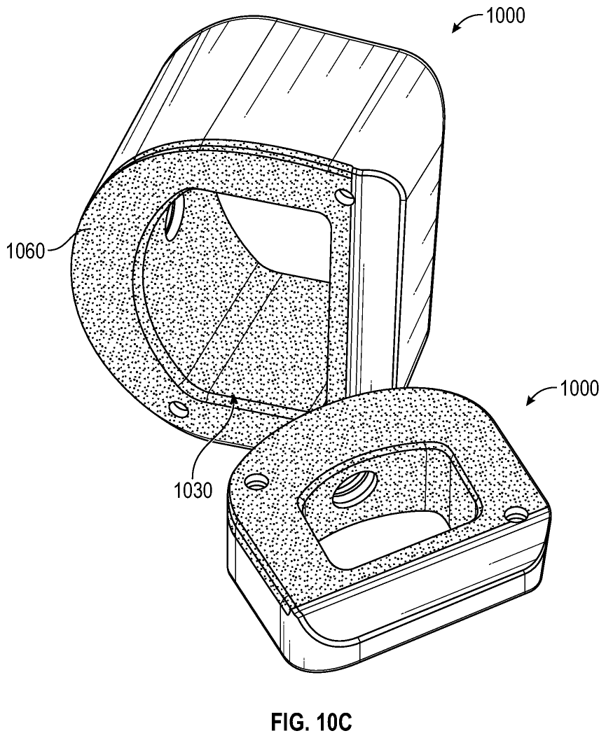

FIG. 10A is a perspective view of an interbody implant, or implant 1000, according to another embodiment. FIG. 10B is an alternative perspective view of the implant 1000 of FIG. 10A. FIG. 10C is another alternative perspective view of the implant 1000 of FIG. 10A, in comparison with another interbody implant such as the implant 100 of FIG. 1A. The implant 1000 may be in a larger size than the implant 100, the implant 600, the implant 700, and the implant 900, and may have a cavity 1030 that similarly promotes fusion between the superior and inferior vertebrae. The implant 1000 may have a porous layer 1060 facing the superior and inferior vertebrae, and optionally, covering the inwardly-facing surfaces of the cavity 1030.

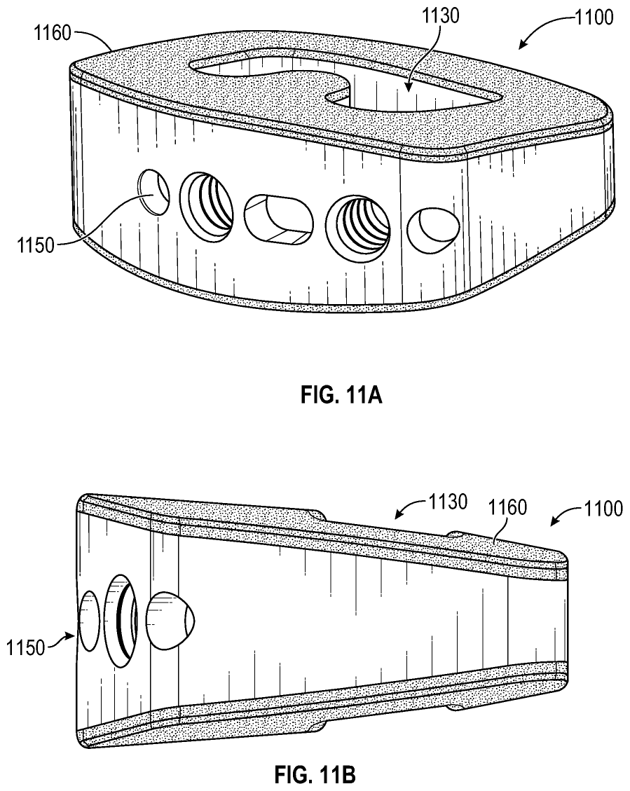

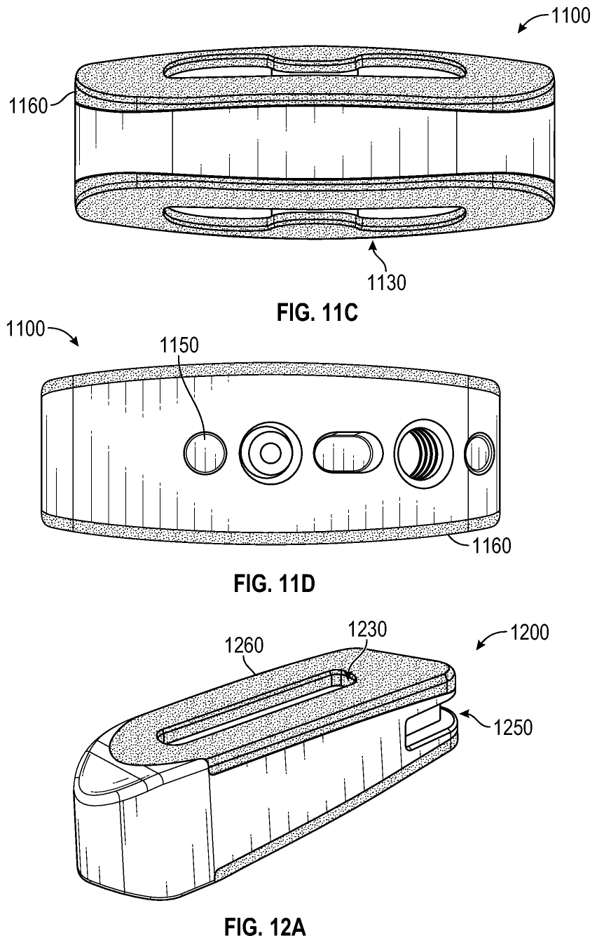

FIG. 11A is a perspective view of an interbody implant, or implant 1100, according to another embodiment. FIG. 11B is a side elevation view of the implant 1100 of FIG. 11A. FIG. 11C is a rear elevation view of the implant 1100 of FIG. 11A. FIG. 11D is a front elevation view of the implant 1100 of FIG. 11A.

The implant 1100 may be an anterior lumbar interbody fusion (“ALIF”) implant designed to be inserted between the superior and inferior vertebrae from an anterior approach. The implant 1100 may have a cavity 1130 that similarly promotes fusion between the superior and inferior vertebrae. The implant 1100 may further have a porous layer 1160 facing the superior and inferior vertebrae, and optionally, covering the inwardly-facing surfaces of the cavity 1130. Additionally, the implant 1100 may have one or more inserter connection features 1150 on the anterior side of the implant 1100. The inserter connection features 1150 may be arranged on one side of the anterior aspect of the implant 1100 to facilitate insertion of the implant 1100 along an antero-lateral approach. The implant 1100 may be designed for use with a bone plate, pedicle screw and rod system, and/or other fixation applied between the superior and inferior vertebrae.

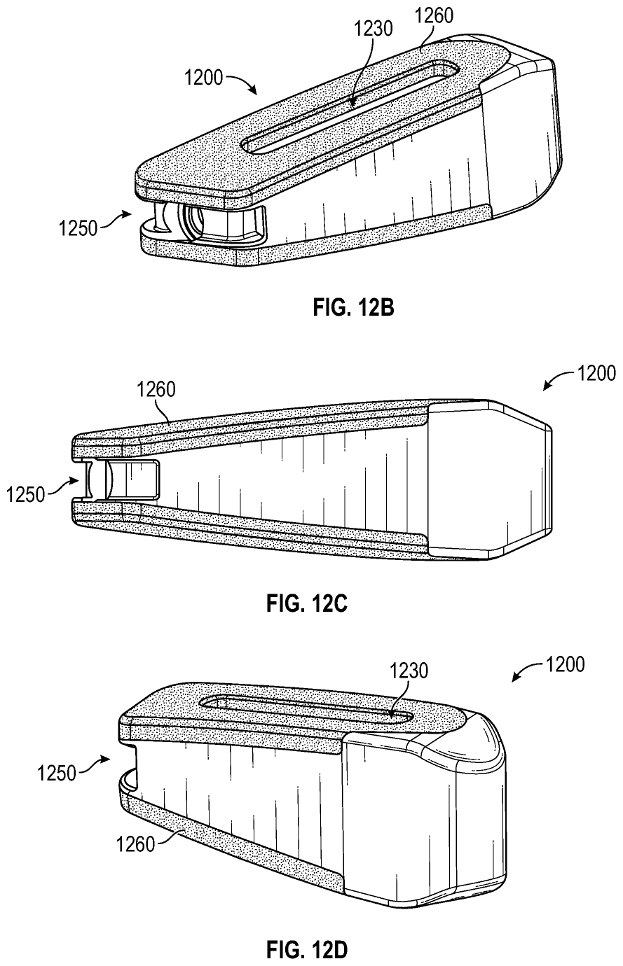

FIG. 12A is a perspective view of an interbody implant, or implant 1200, according to another embodiment. FIG. 12B is an alternative perspective view of the implant 1200 of FIG. 12A. FIG. 12C is a side elevation view of the implant 1200 of FIG. 12A. FIG. 12D is another alternative perspective view of the implant 1200 of FIG. 12A.

The implant 1200 may be a transverse lateral interbody fusion (“TLIF”) implant designed to be inserted between the superior and inferior vertebrae from a transverse lateral approach. The implant 1200 may have a cavity 1230 that similarly promotes fusion between the superior and inferior vertebrae. The implant 1200 may further have a porous layer 1260 facing the superior and inferior vertebrae, and optionally, covering the inwardly-facing surfaces of the cavity 1230. Additionally, the implant 1200 may have one or more inserter connection features 1250 on the posterior side of the implant 1200. The inserter connection features 1250 may facilitate insertion of the implant 1200 along a transverse lateral and/or posterior approach.

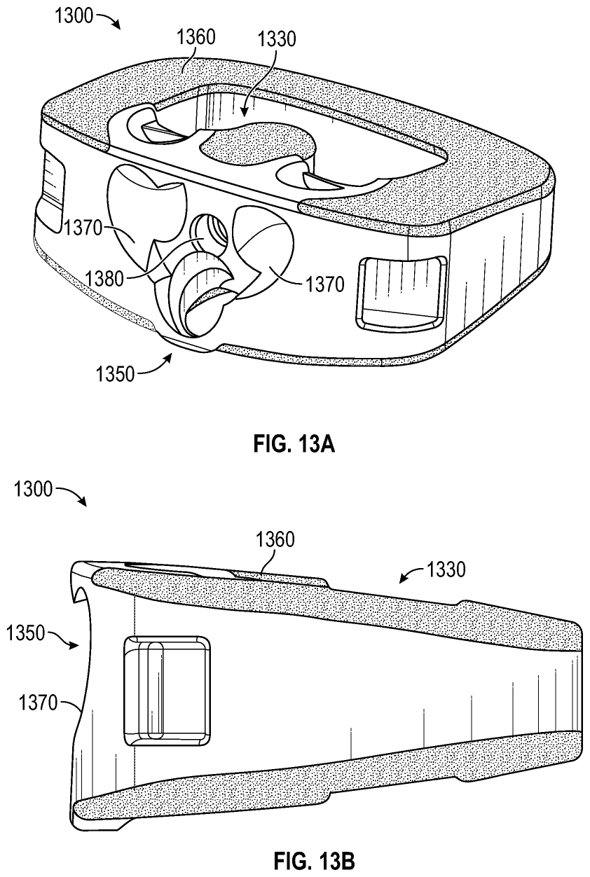

FIG. 13A is a perspective view of an interbody implant, or implant 1300, according to another embodiment. FIG. 13B is a side elevation view of the implant 1300 of FIG. 13A.

The implant 1300 may be a stand-alone anterior lumbar interbody fusion (“ALIF”) implant designed to be inserted between the superior and inferior vertebrae from an anterior approach. The implant 1300 may have a cavity 1330 that similarly promotes fusion between the superior and inferior vertebrae. The implant 1300 may further have a porous layer 1360 facing the superior and inferior vertebrae, and optionally, covering the inwardly-facing surfaces of the cavity 1330. Additionally, the implant 1300 may have one or more inserter connection features 1350 on the anterior side of the implant 1300. The inserter connection features 1350 may be arranged on the anterior aspect of the implant 1300 to facilitate insertion of the implant 1300 along an anterior approach.

The implant 1300 may be designed for use without any additional fixation between the superior and inferior vertebrae. Thus, in addition to the inserter connection features 1350, the implant 1300 may have screw holes 1370 that are angled superiorly and inferiorly to receive bone screws that anchor the implant 1300 directly in the inferior and superior vertebrae. The implant 1300 may further have a hole 1380 that receives an anti-backout device, such as a rotor, that can be deployed to keep the bone screws from backing out of the superior and inferior vertebrae.

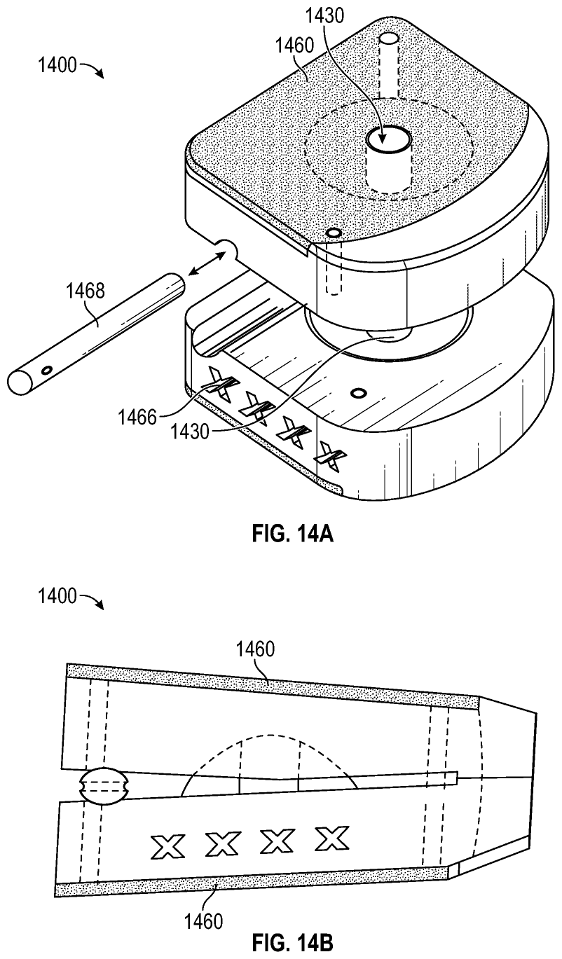

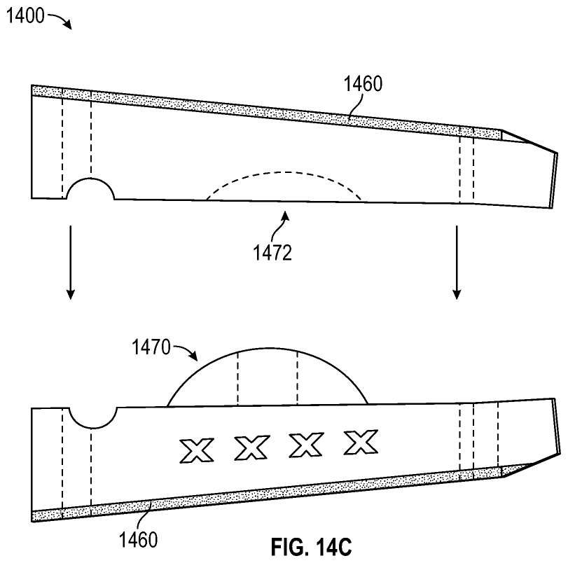

FIG. 14A is a perspective view of an interbody implant, or implant 1400, according to another embodiment. FIG. 14B is a side elevation view of the implant 1400 of FIG. 14A. FIG. 14C is an exploded side elevation view of a partial assembly of the implant 1400 of FIG. 14A.

The implant 1400 may be a stand-alone implant, similar to implant 1300, except that implant 1400 includes two body cores that fit together (see FIG. 14B) to form the implant 1400. In addition to the porous layer 1460 and the cavity 1430, the implant 1400 includes lattice structures 1466 that extend either to a depression in the bottom surface of the implant or through to the other side of the implant 1400, creating bone-growth through-holes in the implant 1400. The lattice structures 1466 may provide additional stability during and/or after fusion of the two vertebrae as they provide additional apertures through which bone growth may occur. Although the lattice structures 1466 are depicted as crisscross or “X” shapes, other lattice and/or aperture shapes may include rectangular, circular, ellipsoidal, square, and combinations thereof.

The implant 1400 further includes a lordotic angle adjustment feature, which is depicted as a pin 1468. Although depicted as a single pin that fits within the corresponding depressions in the two body cores, the pin 1468 may be exchanged with one of multiple different pins each having larger or smaller diameters. For example, if a surgeon wishes to increase or decrease the lordotic angle, they need only use a larger or smaller diameter pin 1468. Although the pin 1468 is depicted as a circular column, other pin shapes are contemplated and included herein. It is also important to note that the lordotic angle adjustment feature may further include a variable thickness porous layer 160 (e.g., thicker layer at the trailing end as compared to the leading end), the connection feature (e.g., convex portion 1470) discussed below, or combinations thereof.

In FIG. 14C, the partial assembly view of the implant 1400 illustrates a connection feature of the implant 1400. The connection feature ensures that the two cores adjustably fit together. For example, the inferior body core of implant 1400 may include a hemispherical, convex portion 1470, while the superior body core of implant 1400 may include a hemispherical (or partially spherical) concave portion 1472. The radius of curvature and/or a diameter of the concave portion 1472 may be less than a radius of curvature and/or diameter of the convex portion 1470, such that a gap exists between the two body cores when the body cores are seated together. This gap, combined with the adjustability provided by the connection feature, allows a surgeon the opportunity to adjust an implant angle (e.g., lordotic angle) after the implant has been inserted between two vertebrae. The pin 1468 may also include a hole through which a surgeon may insert the tip of an instrument to remove the pin after insertion of the implant 1400. By allowing the pin 1468 to remain in between the two body cores during insertion, the implant 1400 is stable (i.e., one body core does not move relative to the other body core) during insertion. By removing the pin 1468 after insertion, the lordotic angle may be controllably adjusted to a desired angle without harming the patient. Rods, pedicle screws, cords, bone screws, or a combination of thereof may be used to stabilize the implant 1400 and the adjacent vertebrae after the desired implant angle is obtained by the surgeon, thereby allowing the vertebrae to fuse at the desired angle. It is noted that ellipsoidal convex and concave shaped connection features, as well as other geometrical shapes, may achieve the same or similar functionality as the hemispherical portions discussed above, each of which are included in the inventive concepts disclosed herein.

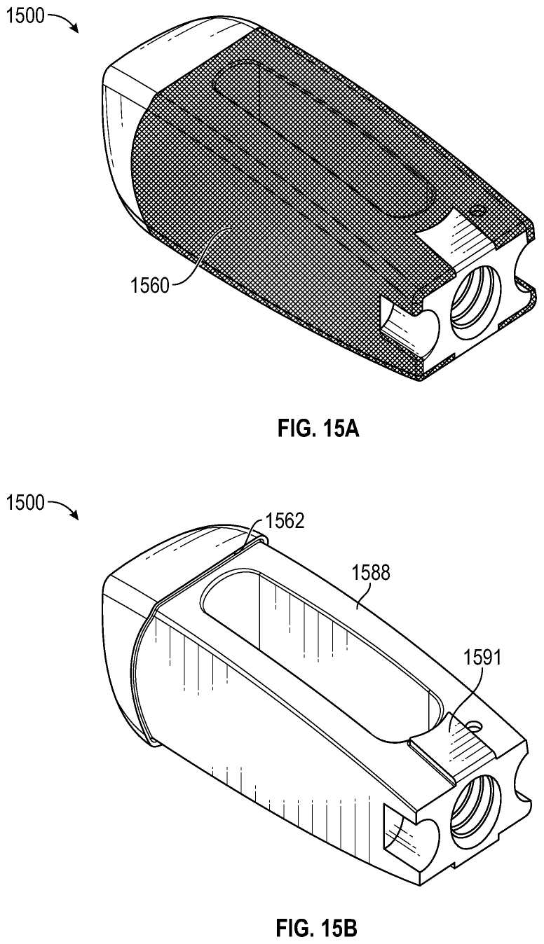

FIG. 15A is a perspective view of an interbody implant, or implant 1500, according to another embodiment. FIG. 15B is a perspective view of the body of the implant 1500 of FIG. 15A. Regarding the term “body” as it is used relative to the implant 1500 (and implants 1600, 1700, and 1800, discussed below), the term means the body core, or the body of the implant 1500 without the porous layer 1560.

The bumper 1562 extends around the perimeter of the body near the leading edge of the implant 1500. The bumper 1562 protects the porous layer 1560 that extends across the lateral sides, superior, and inferior surfaces of the implant 1500, as depicted in FIG. 15A. The bumper may have a linear lip along the superior bumper edge and/or the inferior bumper edge, and a curved (i.e., non-linear) lip along one or both of the lateral sides of the implant 1500. The bone growth cavity of the implant 1500 may have an elongated shape to facilitate bone growth along a portion of the longitudinal length of the implant 1500. The superior and/or inferior surfaces of the implant 1500 may comprise a convex exterior surface 1588. The porous layer 1560 formed on the convex exterior surface 1588 may have a uniform thickness or a variable thickness. The trailing edge of the implant 1500 may include a boss 1591 (e.g., protrusion).

FIG. 16A is a perspective view of an interbody implant, or implant 1600, according to another embodiment. FIG. 16B is a perspective view of the body of the implant 1600 of FIG. 16A.

The leading edge of the implant 1600 includes an immediate transition edge 1686, where the porous material transitions immediately to the material of the solid, non-porous bumper. The inferior and/or superior surface of the body of the implant 1600 includes a flat interior surface 1690. The flat interior surface 1690 does not include a bow or curve in any portion of the surface that extends away from the lip of the bumper up to the trailing edge of the elongated bone growth cavity of the implant 1600. Directly behind and adjacent to the trailing edge of the elongated bone growth cavity is a boss 1691 (e.g., protrusion) of the implant that may interface with the insertion tool or may protect the porous later 1660 at the trailing end of the implant 1600.

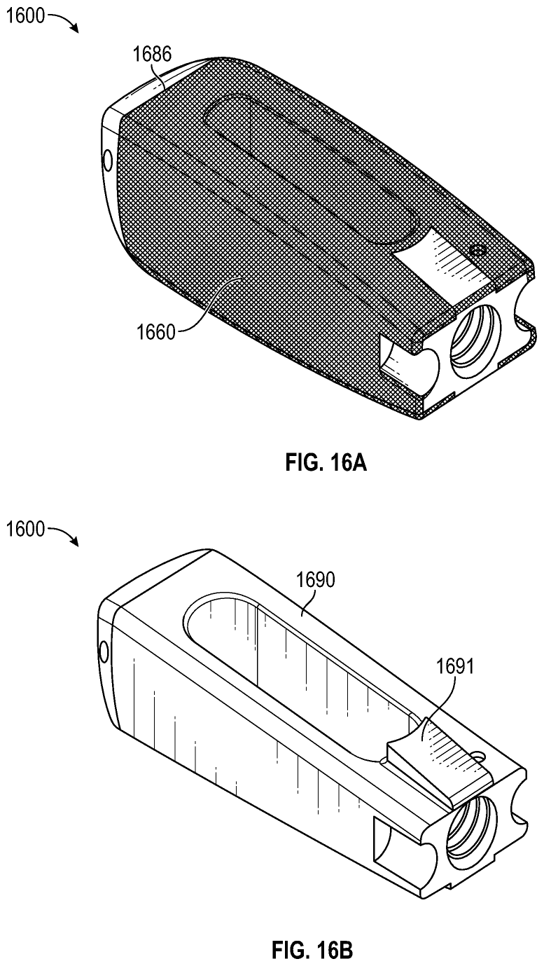

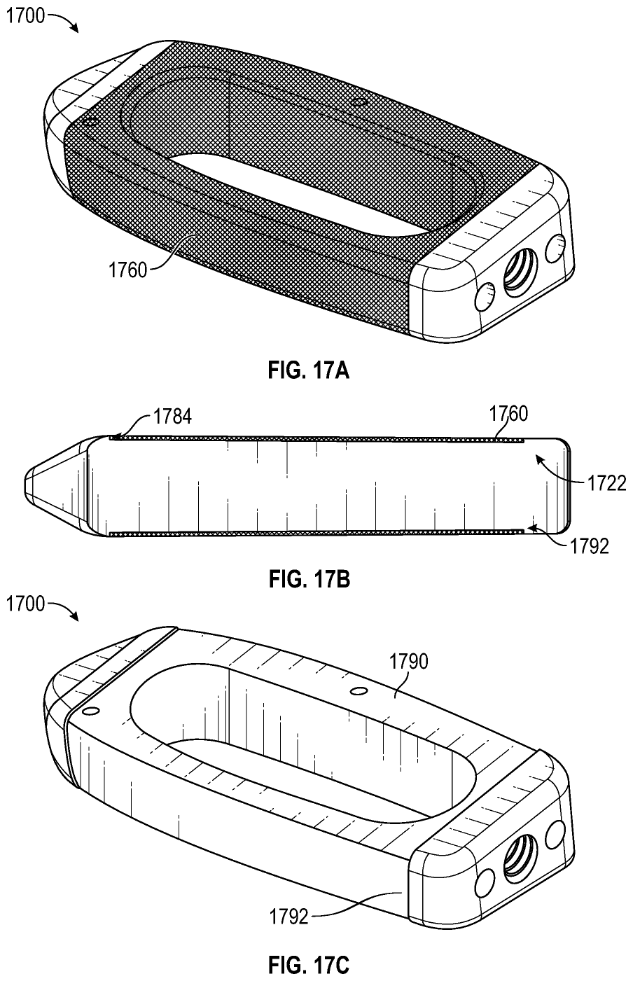

FIG. 17A is a perspective view of an interbody implant, or implant 1700, according to another embodiment. FIG. 17B is a side section view of the implant of FIG. 17A. FIG. 17C is a perspective view of the body of the implant 1700 of FIG. 17A.

The leading end of the implant 1700 may not have linear corners or edges, but rather includes rounded edges and/or corners 1784. The radius of curvature of the rounded edges and/or corners 1784 may be tailorable during manufacturing and may improve the manufacturing process associated with manufacturing the implant 1700. For example, removing a device with rounded corners/edges from a mold may be easier than removing linear devices with linear corners/edges. The shape of the edges and/or corners 1784 may also improve the process of inserting the implant 1700 into the body of the patient (i.e., as compared to inserting implants with linear corners/edges).

The implant 1700 may further include a flat interior surface 1790 and a shoulder 1792 located near the trailing end and on a lateral side 1722 of the implant 1700. The shoulder 1792 may be used as a transition surface near the bumper instead of the lip that was previously discussed. The shoulder 1792 will provide a transitioning edge to the porous layer 1760, instead of an immediate transition from porous material to solid material, as was previously discussed.

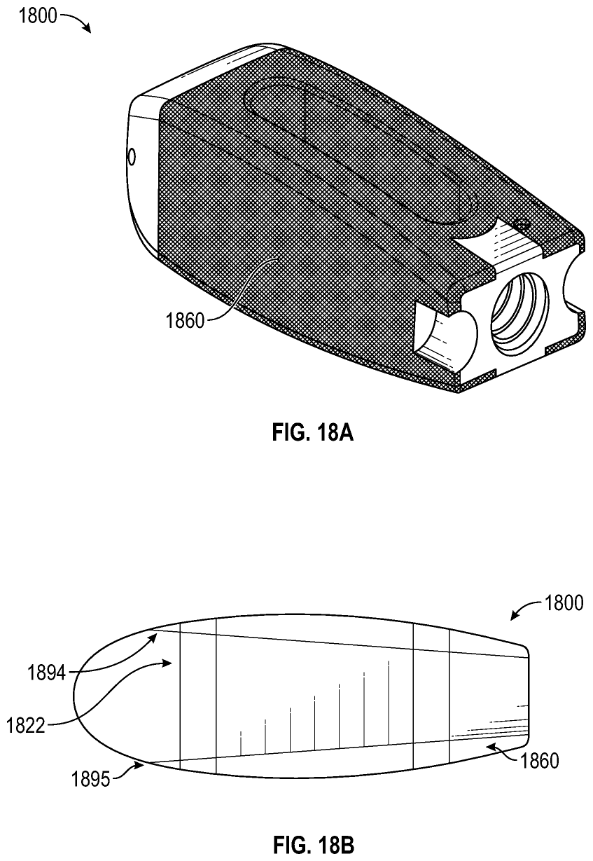

FIG. 18A is a perspective view of an interbody implant, or implant 1800, according to another embodiment. FIG. 18B is a side section view of the lateral side 1822 of the implant 1800. FIG. 18C is a perspective view of the body of the implant 1800 of FIG. 18A.

As depicted in FIG. 18B, the implant may include an interface between the solid body and the porous layer 1860 where a flat interior surface 1894 may face and be secured to at least one of the superior side, the inferior side, the first lateral side, or the second lateral side of the implant 1800. A surface opposite the flat interior surface 1894 may include a convex exterior surface 1895. The flat interior surface 1894 and convex exterior surface 1895 may result in a porous layer with a variable thickness across the width and/or length of the implant. A radius of curvature of the convex exterior surface 1895 of the porous layer may be larger or smaller in different implant embodiments depending on an intended use.

As depicted in FIG. 18C, the implant 1800 includes a solid bumper 1862. Additionally, the superior and/or inferior surfaces of the body of the implant 1800 include one or more flat interior surfaces 1890. Despite the geometry of this/these surface(s), the porous layer 1860 is bowed, as depicted in FIG. 18A. The bowing of the porous layer 1860 is indicated in FIG. 18B, where the bowing is due to the variable thickness of the porous layer 1860. In some embodiments, the bowed porous layer 1860 may be formed on both of the superior and inferior surfaces. In alternative embodiments, the bowed porous layer 1860 may be formed on a single one of the inferior and superior surfaces.

The porous layer 1860 of the implant 1800 further includes transition edges, located at least on the inferior and/or superior surface. The transition edges occur where the material of the surface of the implant 1800 gradually transitions from the material of the porous layer 1860 to the material of the solid, non-porous body core of the implant 1800. Although the transition edges are depicted as abutting the solid bumper 1862 on the superior and inferior surfaces of the implant, in other embodiments the transition edges may be positioned to abut the solid bumper at one or both of the lateral sides 1822.

In some embodiments, at least one of the superior side, the inferior side, the first lateral side, and the second lateral side of the implant 1800 includes a boss 1891 extending through one or more of the porous layers 1860. For example, the boss 1891 may extend fully or partially through the superior portion of the porous layer 1860.

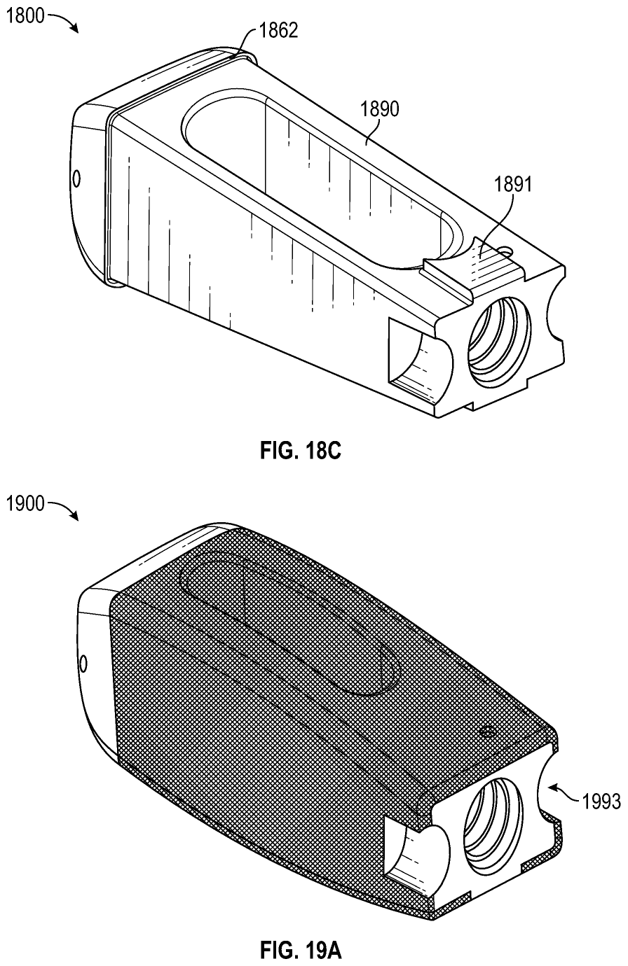

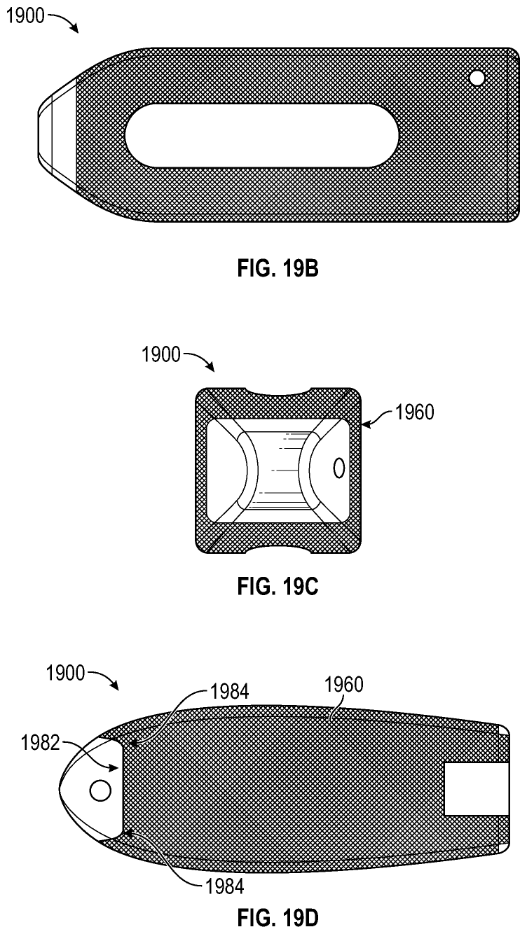

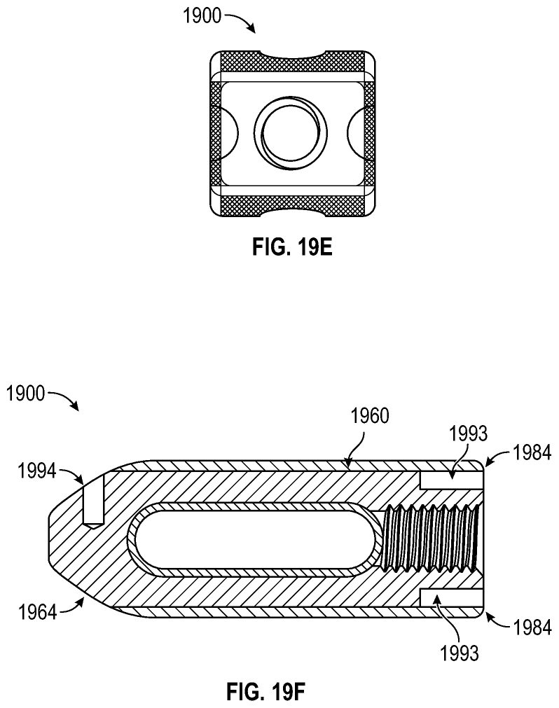

FIG. 19A is a perspective view of an interbody implant, or implant 1900, according to another embodiment. FIG. 19B is a top view of the implant of FIG. 19A. FIG. 19C is a front elevation view of the implant of FIG. 19A. FIG. 19D is a bottom view of the implant of FIG. 19A. FIG. 19E is a rear elevation view of the implant of FIG. 19A. FIG. 19F is a side section view of the implant of FIG. 19A.

In some embodiments, the implant 1900 includes lateral aspects 1993 for accommodating an implant inserter. In other embodiments, the aspects of the implant 1900 for accommodating the implant inserter are not limited to the lateral aspects 1993 but may further include superior and/or inferior aspects. In yet other embodiments, the lateral aspects 1993 may be replaced by the superior and/or inferior aspects that accommodate the implant inserter.

As depicted in FIG. 19D, the porous layer 1960 of the implant 1900 has lateral edges and/or portions of lateral edges that extend toward the leading end, beyond the trailing edge of the solid bumper of the implant 1900. This extension of the porous layer 1960 causes the exterior surface 1982 of the solid bumper to have rounded corners relative to the adjoining exterior surface 1984 of the porous layer 1960 (e.g., rounded corners are formed at the trailing edge of the solid bumper). In some embodiments, the exterior surface 1982 of the solid bumper is flush with the adjoining exterior surface 1984 of the porous layer 1960.

In FIG. 19F, the section view depicts the inferior solid bumper 1964 and/or the superior solid bumper as having the transition edge 1994, where the material of the porous layer 1960 gradually transitions to the solid, non-porous material of the bumper. The implant may further include rounded edges and/or corners at the adjoining exterior surface 1984 at the trailing end of the implant 1900. In some embodiments, the rounded corners at the trailing end of the implant may be due to the solid body having rounded corners. In other embodiments, the rounded corners at the trailing end of the implant 1900 may be due to the adjoining exterior surface 1984 of the porous layer 1960 formed to have rounded corners.

As depicted in FIG. 19D, portions of the porous layer 1960 and/or the underlying body core may be bowed. As depicted in FIG. 19B, portions of the porous layer 1960 and/or the underlying body core may be flat. Additionally, the porous layer 1960 is connected across the entire surface of implant 1900, as depicted in FIGS. 19A and 19C, except where it is interrupted by the insertion features at the trailing end or the solid bumper at the leading end of the implant 1900. The porous layer 1960 may also extend across the exterior and/or vertical surfaces of the implant 1900.

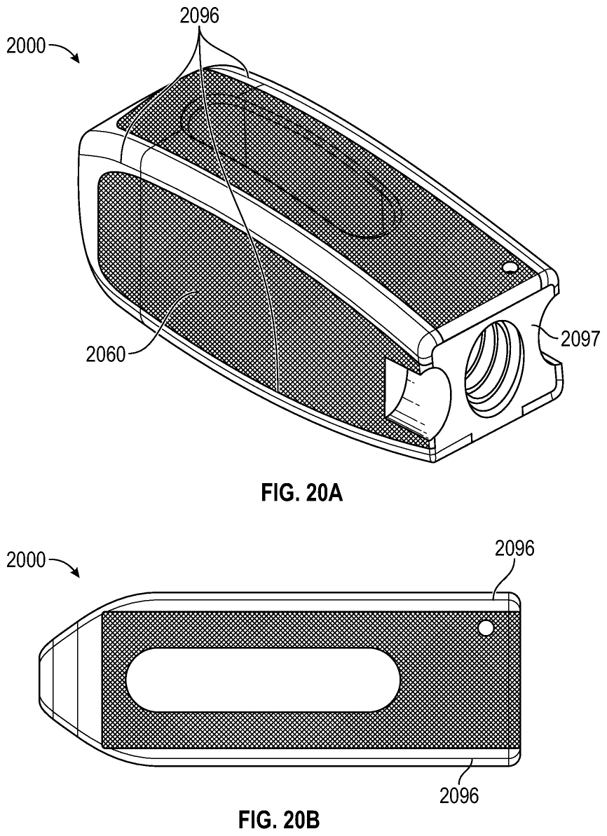

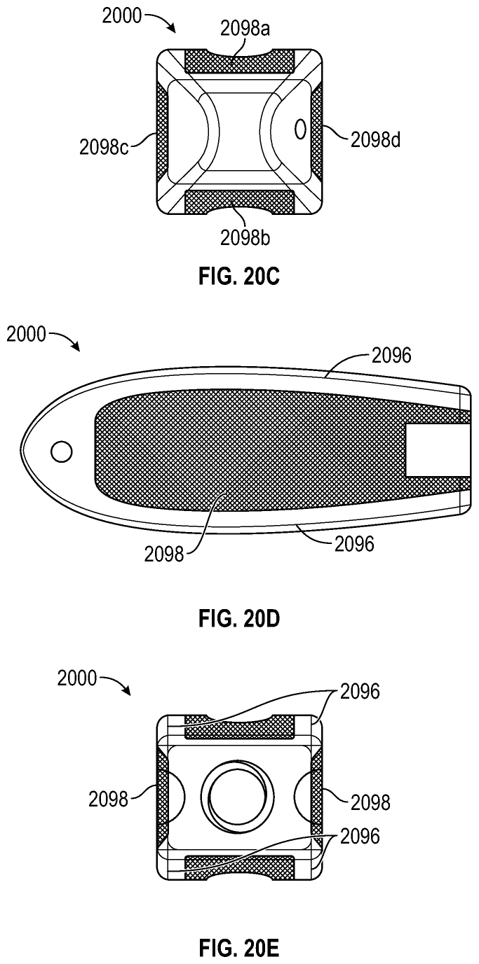

FIG. 20A is a perspective view of an interbody implant, or implant 2000, according to another embodiment. FIG. 20B is a top view of the implant of FIG. 20A. FIG. 20C is a front elevation view of the implant of FIG. 20A. FIG. 20D is a side view of the implant of FIG. 20A. FIG. 20E is a rear elevation view of the implant of FIG. 20A.

FIG. 20A illustrates that the implant 2000 includes solid structures 2096. In some embodiments, the solid structures 2096 are rail-like structures that extend nearly the entire length of the body of the implant 2000 and separate the porous structures 2098 from each other. The solid structures 2096 gradually merge into the solid structure of the bumper. The porous structures 2098 make up the porous layer 2060 of the implant 2000. In some embodiments, the shape of the porous structures 2098 may be determined by the shape of the solid structures 2096. In alternative embodiments, the shape of the solid structures 2096 may be determined by a shape of the porous structures 2098.

In some embodiments, the implant 2000 may include a solid structure 2097 on the trailing end of the interbody implant. The solid structure 2097 may define a non-porous interface that facilitates attachment of the interbody implant 2000 to an implant inserter.

As depicted in FIG. 20C, the porous structures 2098 may include a superior layer 2098a, an inferior layer 2098b, a first lateral layer 2098c, and a second lateral layer 2098d. In some embodiments, the solid structures 2096 may include a portion of the solid body that may be extending longitudinally to separate one or more of the superior layer 2098a, inferior layer 2098b, first lateral layer 2098c, and second lateral layer 2098d. In other embodiments, the solid structures 2096 may include solid structures extending longitudinally between each of the superior layer 2098a, the inferior layer 2098b, the first lateral layer 2098c, and the second lateral layer 2098d.

The material thickness of the porous structures 2098 may be variable, uniform, or both. For example, the flat lateral sides, depicted in FIG. 20B, may include porous structures that have uniform thicknesses attached to the lateral sides, while the bowed superior and/or inferior surfaces depicted in FIG. 20A may have attached porous structures 2098 that have variable material thicknesses.

In FIG. 20C, the leading end, or nose, of the implant 2000 includes rounded or curved surfaces. It is important to note that these rounded or non-linear surfaces of the leading end or nose are not spherical or “bullet-shaped”. This shape of the surfaces of the nose or leading end may further improve the manufacturing process, as it may be easier to round an edge than it is to symmetrically create spherical or bullet shaped noses. Therefore, in some embodiments, the leading edge or nose of the implant 2000 is not spherical or “bullet-shaped”. In alternative embodiments, the leading edge or nose of the implant 2000 may be spherical or “bullet-shaped”.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of the appended claims is not limited to the precise configuration and components disclosed herein. Various modifications, changes, and variations which will be apparent to those skilled in the art may be made in the arrangement, operation, and details of the methods and systems disclosed herein.

What is claimed is:

1. An interbody implant for fusion of a superior vertebra and an inferior vertebra of a patient, the interbody implant comprising:
a body with a solid, unitary structure, the body defining:
a superior side shaped to abut an inferior end plate of the superior vertebra, the superior side defining a first aperture;
an inferior side shaped to abut a superior end plate of the inferior vertebra, the inferior side defining a second aperture;
a leading end;
a trailing end;
a first lateral side;
a second lateral side; and
a bone growth cavity extending between the first aperture and the second aperture; and
one or more porous layers that are secured to the superior side, the inferior side, the first lateral side, and the second lateral side by a homogenous interface between the body and the porous layer.

2. The interbody implant of claim 1, wherein each of the one or more porous layers comprises a web-like scaffold having curved surfaces that define substantially spherical voids, the substantially spherical voids being interconnected at tangent points.

3. The interbody implant of claim 2, wherein the web-like scaffold comprising a material formed from a substantially uniform mixture of osteogenic material and thermoplastic polymer.

4. The interbody implant of claim 3, wherein the body and the one or more porous layers are formed of a thermoplastic polymer and/or a material formed from a substantially uniform mixture of osteogenic material and thermoplastic polymer.

5. The interbody implant of claim 1, wherein the one or more porous layers comprises a single porous layer that extends across the inferior side, the first lateral side, the superior side, and the second lateral side.

6. The interbody implant of claim 1, wherein the one or more porous layers terminate short of the leading end.

7. The interbody implant of claim 6, wherein the leading end comprises a solid bumper positioned to protect the one or more porous layers from abrasion against bone during insertion of the interbody implant between the superior vertebra and the inferior vertebra.

8. The interbody implant of claim 7, wherein the leading end comprises an exterior surface that is flush with adjoining exterior surfaces of the one or more porous layers.

9. The interbody implant of claim 1, wherein at least one of the one or more porous layers comprises a generally uniform thickness.

10. The interbody implant of claim 1, wherein at least one of the one or more porous layers comprises a variable thickness.

11. The interbody implant of claim 10, wherein at least one of the one or more porous layers comprises:
a flat interior surface facing and secured to the superior side, the inferior side, the first lateral side, or the second lateral side; and
a convex exterior surface.

12. The interbody implant of claim 1, wherein:
the one or more porous layers comprise a superior layer secured to the superior side, an inferior layer secured to the inferior side, a first lateral layer secured to the first lateral side, and a second lateral layer secured to the second lateral side; and
the body comprises solid structures extending longitudinally between each of the superior layer, the inferior layer, the first lateral layer, and the second lateral layer.

13. The interbody implant of claim 1, wherein:
the body comprises a solid structure on the trailing end, the solid structure defining a non-porous interface that facilitates attachment of the interbody implant to an implant inserter; and
the one or more porous layers extend to cover superior, inferior, and/or lateral aspects of the trailing end.

14. The interbody implant of claim 1, wherein the one or more porous layers are further secured to inwardly-facing surface of the bone growth cavity.

15. The interbody implant of claim 1, wherein at least one of the superior side, the inferior side, the first lateral side, and the second lateral side comprises a boss extending through the one or more porous layers.

16. The interbody implant of claim 1, further comprising a nano-thick coating of osteogenic material that covers the body and/or the one or more porous layers.

17. An interbody implant for fusion of a superior vertebra and an inferior vertebra of a patient, the interbody implant comprising:
a body with a solid, unitary structure, the body defining:
a superior side shaped to abut an inferior end plate of the superior vertebra, the superior side defining a first aperture;
an inferior side shaped to abut a superior end plate of the inferior vertebra, the inferior side defining a second aperture;
a leading end;
a trailing end;
a first lateral side;
a second lateral side; and
a bone growth cavity extending between the first aperture and the second aperture; and
one or more porous layers that are secured to the superior side and the inferior side;
wherein at least one of the one or more porous layers comprises a variable thickness.

18. The interbody implant of claim 17, wherein at least one of the one or more porous layers comprises:
a flat interior surface facing and secured to the superior side or the inferior side; and
a convex exterior surface.

19. The interbody implant of claim 18, wherein at least one of the one or more porous layers comprises a leading edge at which the variable thickness approaches zero.

20. The interbody implant of claim 17, wherein each of the one or more porous layers is formed of a substantially uniform mixture of osteogenic material and thermoplastic polymer.

21. An interbody implant for fusion of a superior vertebra and an inferior vertebra of a patient, the interbody implant comprising:
a body with a solid, unitary structure, the body defining:
a superior side shaped to abut an inferior end plate of the superior vertebra, the superior side defining a first aperture;
an inferior side shaped to abut a superior end plate of the inferior vertebra, the inferior side defining a second aperture;
a leading end;
a trailing end;
a first lateral side;
a second lateral side; and
a bone growth cavity extending between the first aperture and the second aperture; and
one or more porous layers that are secured to the superior side and the inferior side;
wherein:
the leading end comprises a solid bumper positioned to protect the one or more porous layers from abrasion against bone during insertion of the interbody implant between the superior vertebra and the inferior vertebra;
the body comprises a solid structure on the trailing end, the solid structure defining a non-porous interface that facilitates attachment of the interbody implant to an implant inserter; and
the one or more porous layers extend from the solid bumper to cover superior, inferior, and/or lateral aspects of the trailing end.

22. The interbody implant of claim 21, wherein at least one of the one or more porous layers comprises a variable thickness.

23. The interbody implant of claim 17, wherein each of the one or more porous layers is formed of a substantially uniform mixture of bone growth material and thermoplastic polymer.
